(19) United States
(12) Patent Application Publication
(10) Pub. No.: US 2025/0177384 A1
(43) Pub. Date: Jun. 5, 2025

(54) CHROMANOL COMPOUNDS FOR TREATMENT OR PROPHYLAXIS OF AGEING-ASSOCIATED DISORDERS

(71) Applicant: Sulfateq B.V., Groningen (NL)

(72) Inventors: Robert Henk HENNING, Groningen (NL); Guido KRENNING, Groningen (NL); Adrianus Cornelis VAN DER GRAAF, Groningen (NL); Daniël Henri SWART, Leeuwarden (NL)

(21) Appl. No.: 18/842,075

(22) PCT Filed: Feb. 28, 2023
(86) PCT No.: PCT/NL2023/050095

§ 371 Date: Aug. 28, 2024

(30) Foreign Application Priority Data
Feb. 28, 2022 (NL) 2031091

(51) Int. Cl. A61K 31/496; A61K 31/453; A61P 9/14; A61P 43/00

(52) U.S. Cl. CPC A61K 31/496; A61K 31/453; A61P 9/14; A61P 43/00

(57) ABSTRACT

The invention relates to certain chromanol, quinone or hydroquinone compounds and derivatives thereof for treatment or prophylaxis of aging, or idiopathic ageing-associated disorders such as vascular ageing or reduced renal function. Specifically, the present invention relates to chromanol compounds chosen from 6-hydroxy-2,5,7,8-5 tetramethylchroman-2-yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), 6-hydroxy-2,5,7,8-tetramethylchroman-2-yl)(4-(2-hydroxyethyl)piperazin-1-yl)methanone (SUL-109), or its enantiomers and pharmaceutically acceptable salts thereof.

### FIELD OF THE INVENTION

The invention relates generally to chromanol compounds and derivatives thereof for treatment or prophylaxis of ageing, or ageing-associated disorders. In particular, the invention relates to chromanol compounds and derivatives thereof for treatment or prophylaxis of aging in general, and vascular ageing and/or reduced kidney function due to ageing in particular.

### DESCRIPTION OF THE RELATED ART

The average human life expectancy in most developed countries has doubled in the last 200 years as a consequence of the improved quality of water, food, hygiene, housing and lifestyle, immunization against infectious disease, antibiotics and medical care conditions. However, longer human lives have led to a global burden of late-life disease. About half of human deaths are attributed to ageing-associated disorders or diseases, most prominently cardiovascular disease (CVD), diabetes, chronic obstructive pulmonary disease (COPD), stroke, Alzheimer disease, chronic kidney diseases (CKDs) and cancer, among others (Chang, A., Skirbekk, V., Tyrovolas, S., Kassebaum, N. and Dieleman, J., 2019. Measuring population ageing: an analysis of the Global Burden of Disease Study 2017. The Lancet Public Health, 4(3), pp.e159-e167).

One of the most predominant ageing-associated diseases affecting developed countries is cardiovascular disease (CVD). CVD is triggered by vascular alterations characterized by impaired vasodilation and the overproduction of inflammatory markers (Camici, G., Savarese, G., Akhmedov, A. and Lüscher, T., 2015. Molecular mechanism of endothelial and vascular aging: implications for cardiovascular disease. European Heart Journal, 36(48), pp.3392-3403). Yet, this article also shows the complexity of aging, as many factors are involved.

It is thought that ageing cells and organisms accumulate increased levels of oxidant-damaged nuclear and mitochondrial DNA. The increasing damage to mitochondrial DNA is thought to lead to compromised mitochondrial function and integrity. Consequently, damaged mitochondria trigger the release of more reactive oxygen species (ROS) setting in motion a vicious cycle of increasing DNA damage leading to increased ROS production that leads to more DNA damage. Although the role for oxidants and accumulated DNA damage in the pathogenesis of many age-related diseases is widely accepted, the clinical use of antioxidant therapy has been at best equivocal.

Studies in accelerated aging animal models show that chronic treatment with sGC activator, PDE1 inhibitor or the PDE5 inhibitor, sildenafil, were not able to improve endothelial dependent relaxation (Golshiri, K., Ataei Ataabadi, E., Rubio-Beltran, E., et al., 2021. Selective Phosphodiesterase 1 Inhibition Ameliorates Vascular Function, Reduces Inflammatory Response, and Lowers Blood Pressure in Aging Animals. Journal of Pharmacology and Experimental Therapeutics, 378(2), pp.173-183.; Golshiri, K., Ataei Ataabadi, E., Brandt, R., et al., 2020. Chronic Sildenafil Treatment Improves Vasomotor Function in a Mouse Model of Accelerated Aging. International Journal of Molecular Sciences, 21(13), p.4667).

AT1 receptor blockade with losartan did not have a beneficial effect on the vasomotor function of accelerated aging animal models (Wu, H., van Thiel, B., Bautista-Niño, P., et al., 2017. Dietary restriction but not angiotensin II type 1 receptor blockade improves DNA damage-related vasodilator dysfunction in rapidly aging Ercc1Δ/− mice. Clinical Science, 131(15), pp.1941-1953).

The anticancer drug rapamycin was not able to preserve vasodilator function in accelerated aging animal models (Birkisdóttir, M., Jaarsma, D., Brandt, R., et al., 2021 Aging Cell, 20(2)). Unlike dietary restriction, rapamycin fails to extend lifespan and reduce transcription stress in progeroid DNA repair-deficient mice.

To date, only dietary restriction has successfully improved vasomotor function in accelerated aging animal models. However, dietary restriction as a human lifestyle intervention has limited scope, given the degree of self-restraint required.

There remains a need for new compounds for the effective treatment or prophylaxis of ageing or ageing-associated disorders.

WO2006/105806A1 relates to the use of a complex mixture, comprising at least combination of a statin, a compound suppressing angiotensin production or activity, an anti-inflammatory agent and at least one antioxidant, for the prevention and/or treatment of ageing process itself and the disorders caused by ageing.

WO2015/148522A1 relates to compositions comprising nicotinamide mononucleotide for the treatment of age-associated vascular dysfunction by improving endothelial function and reducing large elastic artery stiffness.

WO2012/116985A1 relates to pharmaceutical compositions comprising at least one renin-angiotensin-aldosterone system inhibitor in a subtherapeutic daily dose and at least one HMG-CoA reductase inhibitor in a subtherapeutic daily dose for use in the prevention, reduction or reversal of arterial aging in apparently healthy subjects.

All these references describe actives with side effects which are unwanted, and/or complex mixtures.

It is an object of the present invention to provide compounds for the treatment or prophylaxis of ageing, and/or ageing-associated disorders, and in particular to provide compounds for the treatment or prophylaxis of vascular aging and/or of reduced kidney function because of aging.

### BRIEF SUMMARY OF THE INVENTION

One or more of the above objects is met by providing certain chromanol, quinone or hydroquinone compounds for use in such treatment.

One or more of the above objects is more in particular met by the present invention by providing compounds according to formula (I), (II), the hydroquinone analogue of formula (II), or a pharmaceutically acceptable salt thereof, for use in the treatment or prophylaxis of aging or idiopathic ageing-associated disorders;

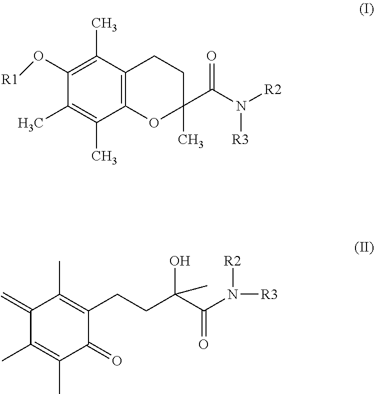

wherein R1 represents a hydrogen or prodrug moiety that can be removed in living tissue
and wherein either
   R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
   or R2 is a hydrogen atom, or an alkyl group with 1-6 carbon atoms, and R3 is an alkyl group, optionally substituted with nitrogen or oxygen, wherein the alkyl group comprises 3-12 carbon atoms, and wherein the alkyl group in R3 comprises one or more non-aromatic cyclic structures that may comprise nitrogen or oxygen atoms in the ring, and may contain linear and/or branched substituted groups, and one or more ethylenic unsaturations.

The present invention in particular provides compounds for use in treatment and prophylaxis of disease conditions associated with a reduced endothelium-dependent NO-cGMP vasodilation. Preferably, the present invention provides compounds for use in the preservation of vascular function through EDHF rescue.

In one preferred embodiment of the invention, treatment of ageing includes increasing the life-span of a mammal, preferably human.

It is understood by the skilled person that any of such treatments is to be considered a medical treatment, and a non-medical treatment is disclaimed for a claim to a medical treatment.

As far as treatment of ageing, and/or improving conditions associated with a reduced endothelium-dependent NO-cGMP vasodilation, and/or the preservation of vascular function through EDHF rescue is not considered a medical treatment, the present invention also provides for the use of the compounds as defined for the increase of life span, improving conditions associated with a reduced endothelium-dependent NO-cGMP vasodilation, and/or the preservation of vascular function through EDHF rescue of a mammal. The mammal preferably is a human. It is understood by the skilled person that any of such treatment is to be considered a non-medical treatment, and a medical treatment is disclaimed for a claim to a non-medical treatment.

For the present invention, the compound according to formula (II) includes the hydrogenated quinone (i.e. the hydroquinone) analogue, although the quinone derivative is preferred in view of stability.

In a preferred embodiment, the nitrogen can be amine, quaternary amine, guanidine or imine and oxygen is hydroxyl, carbonyl or carboxylic acid; and/or oxygen and nitrogen together may form amide, urea or carbamate groups.

In a preferred embodiment of either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms, such as ethylol.

In another preferred embodiment R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

In another preferred embodiment the compound is a compound according to formula II and R2 is a hydrogen atom and R3 comprises a cyclic structure having 4-6 carbon atoms and having one nitrogen atom which ring is unsubstituted or substituted with an alcohol, or alkanol group having 1-4 carbon atoms, such as ethylol, and preferably is optionally substituted with methyl, ethyl, or alcohol substituted methyl or ethyl.

In another preferred embodiment, the compound is a compound according to formula I, R2 is a hydrogen atom and R3 comprises a saturated cyclic structure having 4-7 carbon atoms and having one nitrogen atom, which ring is unsubstituted or substituted with an alcohol, or alkanol group having 1-4 carbon atoms, such as ethylol, and preferably is optionally substituted with methyl, ethyl, or alcohol substituted methyl or ethyl.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

In a most preferred embodiment, the compound is the S-enantiomer of SUL-109, namely S-(6-hydroxy-2,5,7,8-tetramethylchroman-2-yl)(4-(2-hydroxyethyl)piperazin-1-yl)methanone (SUL-138).

### BRIEF DESCRIPTION OF THE DRAWINGS

The features and advantages of the invention will be appreciated upon reference to the following drawings, in which:

FIG. 1: Kaplan-Meier curves displaying the survival of Drosophila treated with varying concentrations of the SUL109: control (purple), 10 uM (blue), 100 uM (green) and 1000 uM (grey). The continuous lines represent females and the dotted lines males.

FIG. 2: Kaplan-Meier curves displaying the survival of Drosophila treated with varying concentrations of the SUL121: control (purple), 10 uM (blue), 100 uM (green) and 1000 uM (grey). The continuous lines represent females and the dotted lines males.

FIG. 3: Effect of chronic SUL 138 treatment on ED relaxation in LM vs. EC-KO mice. Acetylcholine (Ach) dose response curve relative to U46619 pre-constriction. Average is presented as mean±SEM per group. *: significant effect of genotype (p<0.05); #: significant effect of treatment (p<0.05); p=0.054 EC-KO control vs. EC-KO treated.

FIG. 4: Pathway contribution to ACh response in LM vehicle animals. Average is presented as mean±SEM per group. *: significant effect of inhibitor (p<0.05).

FIG. 5: Pathway contribution to ACh response in KO vehicle animals. Average is presented as mean±SEM per group. *: significant effect of inhibitor (p<0.05). FIG. 6: Pathway contribution to ACh response in KO SUL138 treated animals. Average is presented as mean±SEM per group. *: significant effect of inhibitor (p<0.05).

FIG. 7: Albumin and creatinine levels in urine from 22 week old mice. Average is presented as mean±SEM per group. *: significant effect of genotype (p<0.05), (*): p=0.0871.

FIG. 8: Albumin/creatinine ratio in urine and plasma creatinine levels (22 week old mice). Average is presented as mean±SEM per group. *: significant effect of genotype (p<0.05).

FIG. 9: Creatine levels in blood plasma (22 week old mice). Average is presented as mean±SEM per group. *: significant effect of genotype (p<0.05).

FIG. 10: Creatinine levels in 24 h urine from 21 week old mice. Average is presented as mean±SEM per group. *: significant effect of genotype (p<0.05), (*): p=0.088.

### DETAILED DESCRIPTION OF THE INVENTION

One or more of the objects of the present invention, to provide compounds for the treatment or prophylaxis of ageing, and/or ageing-associated disorders is met by providing compounds according to formula (I) or (II), as shown above, or a pharmaceutically acceptable salt thereof.

R1 can be a substituent that is easily removed in the human body, such that the compound is a prodrug. R1 can be for example an amino acid derivative or ester derivative, and generally has a molecular weight lower than 100 dalton.

In a preferred embodiment, R1 in formula (I) is hydrogen or forms together with the 6-oxygen an ester group with 2-6 carbon atoms. The ester can comprise one or more ether or alcohol groups. Suitable esters are acetate, butyrate, 3-hydroxy butyrate and the like.

In a preferred embodiment of either compounds according to formula (I) or according to formula (II), R2 and R3 together with the N atom to which they are attached form a saturated ring having 3-6 carbon atoms and incorporating one additional N atom, which may be substituted with 1-4 carbon atoms that may comprise an oxygen, carboxylic acid or amine group.

More preferably, R2 and R3 together with the N atom to which they are attached form a 5-7 membered ring comprising one additional amine group, which ring is optionally substituted with methyl, ethyl, or alcohol substituted methyl or ethyl.

In another preferred embodiment, R2 is a hydrogen atom and R3 comprises a cyclic structure having 3-6 carbon atoms and having one nitrogen atom.

More preferably, R2 is a hydrogen atom, and R3 comprises a 5-7 membered ring comprising one additional amine group, which ring is attached to the amide-nitrogen, and which ring is optionally substituted with methyl, ethyl, or alcohol substituted methyl or ethyl.

In either case, the ring (the cyclic structure formed by R2 and R3, or of R3 alone) may be unsubstituted or substituted with an alkyl having 1-4 carbon atoms, alcohol, or alkanol group having 1-4 carbon atoms, such as ethylol.

In a preferred embodiment according to the invention, the compound either according to formula (I) or according to formula (II) has a molecular weight lower than 500 Da.

In a preferred embodiment, the compound for use according the present invention is a chromanol compound according to formula I.

Certain chromanol compounds have been described in WO2014/098586. The compounds described in detail have abbreviations, referring to SUL-XXX (XXX being a 2 or 3 digit number). Many of these compounds are racemic mixtures, although some enantiomers have been tested as well. Suitable methods to prepare chromanol compounds according to the present invention are described in WO2014/098586 or WO2014/011047.

WO 2017/060432 A1 discloses amide-derivatives of 2-hydroxy-2-methyl-4-(3,5,6-trimethyl-1,4-benzoquinon-2-yl)-butanoic acid and methods of making such compounds.

Hydrogenated quinone derivatives can be easily prepared by hydrogenation of the quinone structure.

According to yet another preferred embodiment, the compound is either (6-hydroxy-2,5,7,8-tetramethylchroman-2y1)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

In a most preferred embodiment, the compound is the S-enantiomer of SUL-109,namely S-(6-hydroxy-2,5,7,8-tetramethylchroman-2-yl)(4-(2-hydroxyethyl)piperazin-1-yl)methanone (SUL-138).

The counterion in the pharmaceutically acceptable salt can be a counterion as known in the art. Preferably, the compounds have at least one basic nitrogen, an amine, which can be protonated. The counterion preferably is a halogen such as chloride, sulphate, citrate, formate or the like, and most preferably chloride.

The compounds are effective as a racemic mixture or in a substantially pure enantiomeric form. The compounds have one or more chiral centers, generally one or two.

Preferably, the compound is a substantially enantiomerically pure compound. Substantially enantiomerically pure is about 95% enantiomeric excess or more, more preferably about 98% enantiomeric excess, and most preferably about 99% or more enantiomeric excess. Also, in case the compound contains more than one chiral center, these amounts apply.

The compounds are preferably used in effective amounts, to achieve treatment or prophylaxis of ageing or ageing-associated disorders.

The wording treatment or prophylaxis includes amelioration of the symptoms of ageing and/or reduction in progress of ageing, including improvement of vascular and renal function.

The term ‘treatment’ encompasses reduction in progress of the disorder and/or improvement in symptoms of the disorder.

In one preferred embodiment of the invention, treatment of ageing includes increasing the life-span of a mammal, preferably human.

Preferably, the compounds according to the invention are for use of treatment or prophylaxis of ageing-associated disorders in mammals, wherein the mammal is preferably human.

Ageing related disorders include vascular ageing, reduced kidney function, loss of memory, reduced lung-function and the like. These disorders are idiopathic, or just ageing related, but have no specific underlying disease like asthma, COPD, Alzheimer or the like. Hence, administering the compounds of the present invention increase the quality of life and/or life span of the ageing mammals.

It is understood by the skilled person that any of such treatments is to be considered a medical treatment, and a non-medical treatment is disclaimed for a claim to a medical treatment.

As far as any of the forgoing treatment, like for example treatment of ageing, and/or improving conditions associated with a reduced endothelium-dependent NO-cGMP vasodilation, and/or the preservation of vascular function through EDHF rescue is not considered a medical treatment, the present invention also provides for the use of the compounds as defined for the increase of life span, improving conditions associated with a reduced endothelium-dependent NO-cGMP vasodilation, and/or the preservation of vascular function through EDHF rescue of a mammal. The mammal preferably is a human. It is understood by the skilled person that any of such treatment is to be considered a non-medical treatment, and a medical treatment is disclaimed for a claim to a non-medical treatment.

In a more preferred embodiment according to the invention, the compound the compound either according to formula (I) or according to formula (II) is for use for treating or prophylaxis of vascular ageing.

The present invention provides compounds for use in treatment or prophylaxis of ageing disorders associated with a deterioration of the mitochondrial function and health.

The present invention provides compounds for use in treatment and prophylaxis of disease conditions associated with a reduced endothelium-dependent NO-cGMP vasodilation. Preferably, the present invention provides compounds for use in the preservation of vascular function through EDHF rescue.

Even more preferably, the compound the compound either according to formula (I) or according to formula (II) is for use for treating or prophylaxis of cardiovascular disease (CVD).

In a further embodiment, the compound the compound either according to formula (I) or according to formula (II) is for use for treating or prophylaxis of reduced kidney function because of ageing.

Effects generally are observed with amounts of about 1 μM in body fluid, but preferably higher amounts are used. Preferred amounts are concentrations in vivo or in vitro of about 10 μM or higher, more preferably about 20 μM or higher. Generally, a concentration in human of about 200 μM or lower should be sufficient and safe.

For human use, this would mean—assuming a 30 L distribution volume, 100% availability and a concentration of about 1 μM—a dosage of about 10 mg or more. Preferred amounts would result in a concentration of about 10 μM—for which a dosage of about 100 mg or more would be suitable. Hence, preferably, dosage forms of about 20 mg or more, preferably 50 mg or more, preferably 100 mg or more are suitable.

Generally, solid, oral dosage forms contain as a maximum about 500 mg compound, preferably about 450 mg or less, to allow for excipients.

With parenteral administration, such as for example i.v. other liquid forms of administration, larger amounts can be administered.

Examples of dosages which can be used are an effective amount of the compounds of

the invention of a dosage of 0.2 mg/kg or higher, such as preferably within the range of about 1 mg/kg to about 100 mg/kg, or within about 2 mg/kg to about 40 mg/kg body weight, or within about 3 mg/kg to about 30 mg/kg body weight, or within about 4 mg/kg to about 15 mg/kg body weight. Compounds of the present invention may be administered in a single daily dose, or the total daily dosage may be administered in divided dosage of two, three or four times daily.

The prophylaxis or treatment of medical or non-medical ageing or ageing related disorders generally requires chronic administration of the active compound, i.e. administration of the active over a long period of time. Preferably, the chronic administration generally will comprise administration of the active compound for at least 2 months, preferably for at least 4 months, and even more preferred for more than 6 months. In a further preferred embodiment, the administration will comprise administration of the active compound over a period of 1, 2, 3, 4, 5 or 6 years or more, like up to 10, 20 or 30 years. Administration of the active may be over a period of 60 years or less.

The treatment over a relatively long period of several months, several years or even longer may comprise daily administration of one or two or more orally taken dosage forms, like tablets or liquids. Preferably, one oral dosage form is administered once daily. However, alternative administration regimens can be efficacious, like every other day, the use of drug holidays such as for example three weeks daily administration and one week no active administered, for at least 4 or 6 months or longer like several years.

Thus, according the present invention, chronic treatment comprises administration of the active over several months or longer, preferably several years or longer, wherein an effective amount of compound is administered for the whole of the period. As explained above, the compound is administered regularly or intermittently during such period.

The compounds described herein can be formulated as pharmaceutical compositions by formulation with additives such as pharmaceutically or physiologically acceptable excipients carriers, and vehicles.

Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidone, low melting waxes, and the like, as well as combinations of any two or more thereof. Other suitable pharmaceutically acceptable excipients are described in “Remington's Pharmaceutical Sciences,” Mack Pub. Co., New Jersey (1991).

A pharmaceutical composition preferably comprises a unit dose formulation, where the unit dose is a dose sufficient to have a therapeutic effect. The unit dose may be a dose administered periodically in a course of treatment or suppression of a disorder.

The compounds of the invention may be administered enterally, orally, parenterally, sublingually, by inhalation (e. g. as mists or sprays), rectally, or topically in dosage unit formulations containing conventional nontoxic pharmaceutically or physiologically acceptable carriers, adjuvants, and vehicles as desired. The term parenteral as used herein includes subcutaneous injections, intravenous, intramuscular, intratarsal injection, or infusion techniques. The compounds are mixed with pharmaceutically acceptable carriers, adjuvants, and vehicles appropriate for the desired route of administration.

Generally, oral administration is a preferred route of administration, and formulations suitable for oral administration are preferred formulations.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavouring, and perfuming agents.

The amount of active ingredient that may be combined with the carrier materials to produce a single dosage form will vary depending upon the host to which the active ingredient is administered and the particular mode of administration. The unit dosage chosen is usually fabricated and administered to provide a defined final concentration of drug in the blood, tissues, organs, or other targeted region of the body. The effective amount for a given situation can be readily determined by routine experimentation and is within the skill and judgment of the ordinary clinician or skilled person.

The present invention will be further illustrated using the examples below. In the examples, reference is made to figures.

### EXAMPLES

### Example 1—Study of the Effect of SUL Compounds on the Lifespan of Drosophila Model

### Experimental Methodology

### Fly Stocks and Food Supplementation

W1118 flies were bred and housed at 25° C. with a 12 h:12 h light/dark-cycle. Flies were kept in vials containing approximately 5 mL of Bloomington food consisting of sucrose, yeast suspension, agar, Nigapin and fresh yeast. Flies kept as a stock were flipped to fresh vials weekly. Food was stored at 18° C. and warmed at room temperature before use.

SUL109 and SUL121 were used to assess the effect of chromanol compounds on the lifespan of the fly stocks. The compounds were independently tested. Either SUL109 or SUL121 were added to the food to a final concentration of 10 μM, 100 μM and 1000 μM.

### Drosophila Lifespan

W1118 flies were anesthetized in small groups using CO₂. Subsequently, flies were collected at a density of 5 males and 5 females per tube.

At day 10 and 11 eclosing adults were anesthetized and housed at a density of 20 males and females per tube. Ten vials were used per study condition (total of 100 males and 100 females).

During the lifespan, flies were transferred onto new vials containing fresh food 3 times a week, and the number and sex of dead flies was recorded.

### Results

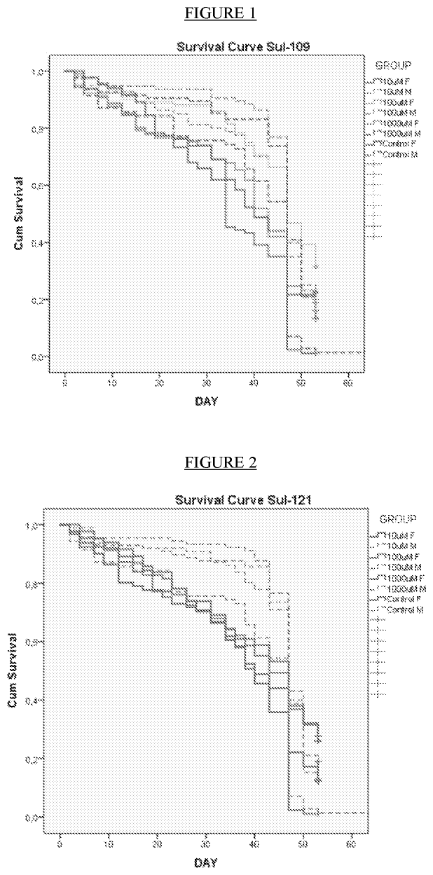

W1118 flies were exposed to different concentrations of chromanol-based compounds (SUL109 and SUL121) in their food since larvae state until the moment they died. Results for both SUL components show flies that were exposed to the higher doses of the compounds have a better survival than the controls (see FIG. 1 for SUL109; FIG. 2 for SUL121).

In the current experimental set up, the effect of two of those compounds, SUL109 and SUL121 on the lifespan of Drosophila was tested. Results show an increased lifespan associated with the treatment with SUL compounds.

### Example 2—Study of the Effect of SUL Compounds on the Vascular and Renal Function of Ercc1^{-/-} (EC-KO) Mice Models

### Background

The effectiveness of the compounds according to the invention for treatment or prophylaxis of ageing-associated diseases was studied in Ercc1^{-/-} (EC-KO) mice models.

Ercc1^{-/-} (EC-KO) mice models possess a Endothelial cell (EC)-specific deletion of DNA repair endonuclease Ercc1, which leads to accelerated vascular aging features, marked by reduced endothelium-dependent NO-cGMP vasodilation at 22 weeks of age.

Therefore, EC-KO mice are excellent animal models to study ageing-associated diseases, in particular vascular ageing.

### Experimental Methodology

### Study Design

EC-KO mice and corresponding littermates (LM) received either SUL138 treated (30 mg SUL138/kg BW/day) or vehicle treated (0.015% [v/v] ethanol) chow for 8 weeks (14-22 weeks of age).

At the age of 21 weeks, animals were put in metabolic cages. After 10 h of acclimatization, 24 h urine was collected. At the age of 22 weeks, mice were euthanized, urine, blood plasma and tissue were collected.

### Vascular Function

Segments of thoracic aorta were isolated and mounted in wire-myograph containing Krebs Henseleit buffer (at 37° C., oxygenated with 95% O2 and 5% CO2) to assess endothelium-dependent (ED) relaxation.

Contraction of the thoracic aorta isolates was induced with 100 mM KCl (final concentration) to evaluate vessel viability.

A stable synthetic analog of the endoperoxide prostaglandin PGH2, namely U46619, was used for pre-constriction. Thoracic aorta segments were washed and subsequently pre-constricted with U46619 (30-60 nM final concentration; 75-100% of the response to 100 mM KCl), followed by a dose response curve with ED vasodilator acetylcholine (ACh) (10⁻⁹-10⁻⁵ M final concentration).

To evaluate the contribution of different pathways to vasodilator response, segments were incubated with inhibitors for 15 minutes prior to pre-constriction with U46619. L-NAME (10⁻⁴ M) was added to assess nitric oxide (NO) contribution to vasodilator response. L-NAME, apamin (100 nM) and TRAM-34 (10 μM) were added to assess ED hyperpolarization factor (EDHF) contribution to vasodilator response.

The acute effect of SUL138 was evaluated by pre-incubating with 100 μM for 30 minutes.

### Renal Function

Creatine levels in urine were measured in the samples collected at the age of 21 weeks and at the time euthanasia (22 weeks). QuantiChrom Creatinine Assay Kit (1:2 sample dilution) was used according to manufacturer's instructions.

Albumin levels in urine were measured in the samples collected at the time euthanasia (22 weeks). Mouse albumin ELISA kit ab108792 (1:400 sample dilution) was used according to manufacturer's instructions.

Blood plasma creatinine was measured in the samples collected at the time euthanasia (22 weeks). Samples were undiluted. QuantiChrom Creatinine Assay Kit (1:2 sample dilution) was used according to manufacturer's instructions.

### Results

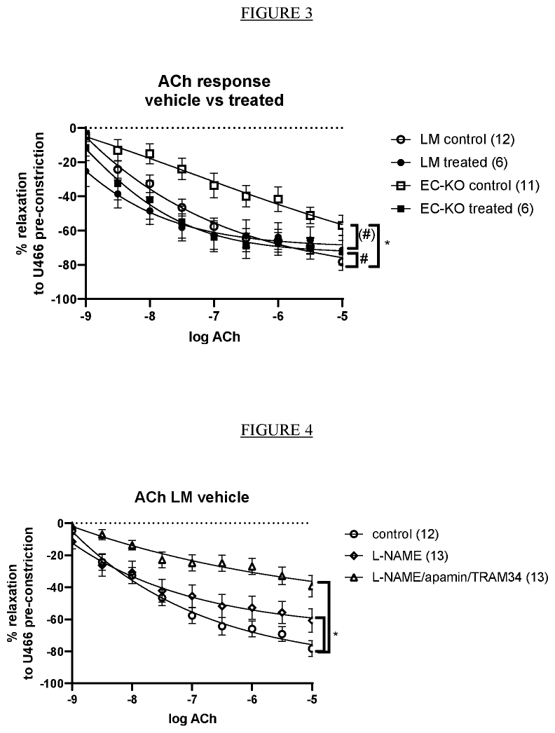

FIG. 3 shows the effect of chronic treatment of LM and EC-KO mice with SUL138. Chronic treatment with SUL138 restored ED relaxation in EC-KO mice compared to LM control levels.

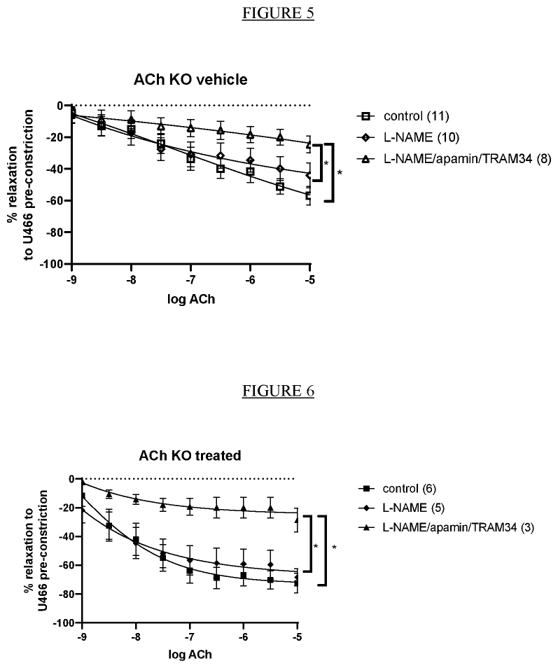

FIG. 4 shows the pathway contribution to ACh response in LM control (vehicle treated) mice. It is demonstrated that ED relaxation in LM control mice is partly NO (see L-NAME curve in FIG. 4) and partly EDHF (see L-NAME/apamin/TRAM34 curve in FIG. 4) driven.

The pathway contribution to ACh response was also tested in EC-KO mice (vehicle treated). Result curves are shown in FIG. 5. The dysfunctional endothelium in the vehicle treated EC-KO led to a shift towards mainly EDHF-driven ED relaxation (see L-NAME curve in FIG. 5).

The results of the pathway contribution to ACh response in EC-KO mice treated with SUL138 showed that the effect of such chronic treatment results from an increased EDHF-driven ED relaxation, as shown in FIG. 6 (see L-NAME curve).

However, restoration of ED relaxation in EC-KO mice only occurred after chronic treatment with SUL138. There is no effect on ED relaxation following acute treatment of mice with SUL138.

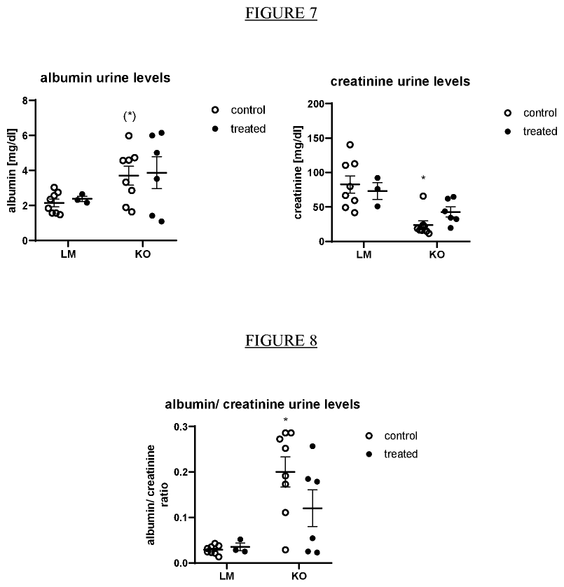

FIG. 7 shows the albumin and creatin levels in urine in 22 week old mice. Vehicle treated EC-KO mice displayed a trend of albuminuria and decreased creatinine filtration compared to vehicle treated LM at the age. The results show that chronic treatment with SUL138 significantly improved creatinine filtration in EC-KO mice.

FIG. 8 shows the albumin/creatine ratio in urine and blood plasma in 22 week old mice. It is shown that albumin/creatinine ratio in urine was significantly elevated in vehicle treated EC-KO mice compared to vehicle treated LM mice. Chronic treatment with SUL138 led to a significant improvement of albumin/creatinine ratios in EC-KO mice.

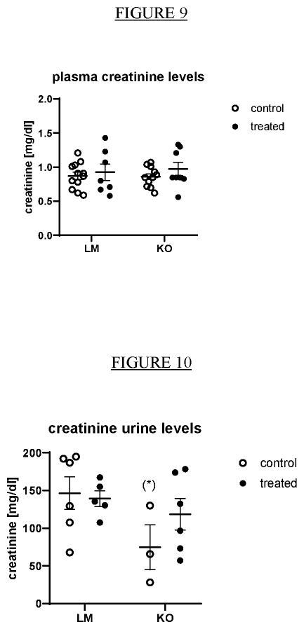

No effects could be however identified in the blood plasma creatinine content in vehicle treated EC-KO mice compared to vehicle treated LM mice (FIG. 9).

FIG. 10 shows the creatine levels in 24 h urine from 21 weeks old mice. Results obtained from the analysis of urine taken at 21 weeks of age, namely 24 h urine, showed the same trend of decreased creatinine filtration in vehicle treated EC-KO mice compared to vehicle treated LM mice. Such trend was significantly improved by chronic treatment of EC-KO mice with SUL138.

### Conclusion

It is often observed that EDHF replaces NO in models of progressive endothelial dysfunction. In the present study, Ercc1-deficient mouse models are strongly aged at 16 weeks of age, such that they have partially lost EDHF and totally lost NO.

Our results show that SUL138 delays endothelial aging. The increase of EDHF instead of NO following chronic treatment with SUL138 suggests a compensation for loss of NO. Alternatively, chronic treatment with SUL138 might increase EDHF as a direct effect.

In summary, chronic treatment with SUL138 resulted in full preservation of vascular function through EDHF rescue in isolated tissue from Ercc1-deficient mouse models.

Moreover, chronic treatment with SUL138 also resulted in the protection of renal function in Ercc1-deficient mouse models.

To date, only dietary restriction was able to improve vasomotor function in Erccl-deficient, accelerated aging, mouse models (Wu et al., 2017, CI Sci). Contrary to the effect of other drugs in endothelial dysfunction models, SUL138 results in an attenuation of the accumulation of persistent DNA damage, similar to the proven effects of dietary restriction.

Thus, the invention has been described by reference to certain embodiments discussed above. It will be recognized that these embodiments are susceptible to various modifications and alternative forms well known to those of skill in the art.

Further modifications in addition to those described above may be made to the structures and techniques described herein without departing from the spirit and scope of the invention. Accordingly, although specific embodiments have been described, these are examples only and are not limiting upon the scope of the invention.

1. Compound according to formula (I) or (II), the hydroquinone analogue of formula (II), or a pharmaceutically acceptable salt thereof for use in the treatment or prophylaxis of ageing or idiopathic ageing-associated disorders; 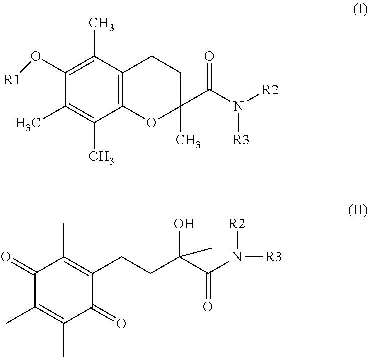
wherein R1 represents a hydrogen or prodrug moiety that can be removed in living tissue
and wherein either
R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
or R2 is a hydrogen atom, or an alkyl group with 1-6 carbon atoms, and R3 is an alkyl group, optionally substituted with nitrogen or oxygen, wherein the alkyl group comprises 3-12 carbon atoms, and wherein the alkyl group in R3 comprises one or more non-aromatic cyclic structures that may comprise nitrogen or oxygen atoms in the ring, and may contain linear and/or branched substituted groups, and one or more ethylenic unsaturations.

2. Compound for use according to claim 1, wherein R1 is hydrogen or forms together with the 6-oxygen an ester group with 2-6 carbon atoms.

3. Compound for use according to any one of claims 1-2, wherein the nitrogen in R2 and/or R3 can be amine, quaternary amine, guanidine, or imine and the oxygen in R2 and/or R3 can be hydroxyl, carbonyl or carboxylic acid; and/or oxygen and nitrogen in R2 and/or R3 together can form amide, urea or carbamate groups and wherein preferably the compound according formula (I) or formula (II) has a molecular weight lower than 500 Da.

4. Compound for use according to any one of claims 1-3, wherein in either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms.

5. Compound for use according to any one of claims 1-4, wherein the compound is a compound according to formula I.

6. Compound for use according to claim 5, wherein R2 and R3 together with the N atom to which they are attached form a 5-7 membered ring comprising one additional amine group, which ring is optionally substituted with methyl, ethyl, or alcohol substituted methyl or ethyl.

7. Compound for use according to any one of claims 1-3, wherein R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group, and preferably, wherein the compound is a compound according to formula I and wherein R2 is a hydrogen atom and R3 comprises a cyclic structure having 4-6 carbon atoms and having one nitrogen atom which ring is optionally substituted with methyl, ethyl, or alcohol substituted methyl or ethyl.

8. Compound for use according to claim 1, wherein the compound is (6-hydroxy-2,5,7,8-tetramethylchroman-2-yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13) or (6-hydroxy-2,5,7,8-tetramethylchroman-2-yl)(4-(2-hydroxyethyl)piperazin-1-yl)methanone (SUL-109) or a pharmaceutically acceptable salt thereof, as a racemic mixture or as one of its enantiomers.

9. Compound for use according to claim 8, wherein the compound is the S-enantiomer of SUL-109: S-(6-hydroxy-2,5,7,8-tetramethylchroman-2-yl)(4-(2-hydroxyethyl)piperazin-1-yl)methanone (SUL-138) or a pharmaceutically acceptable salt thereof.

10. Compound for use according to any of the preceding claims, wherein the use is for the treatment or prophylaxis of vascular aging, for delaying ageing, and/or for the treatment or prophylaxis of reduced kidney function due to aging.

11. Compound for use according to any one of the preceding claims, wherein the compound is administered chronically, preferably comprising administration of the active over several months or longer, more preferably several years or longer, wherein an effective amount of compound is administered for substantially the whole of the period.

12. Compound for use according to any one of the preceding claims, wherein the treatment and prophylaxis of disease conditions are associated with a reduced endothelium-dependent NO-cGMP vasodilation.

13. Compound for use according to any one of the preceding claims, wherein the treatment and prophylaxis of disease conditions are associated with the preservation of vascular function through EDHF rescue.

14. Use of a compound as described in any one of claims 1-9 for delaying ageing of a mammal, preferably a human, wherein a medical treatment is disclaimed.

15. Use according to claim 14, wherein the delay of aging is associated with improving conditions associated with a reduced endothelium-dependent NO-cGMP vasodilation, and/or the preservation of vascular function through EDHF rescue.
